SEQUENCE LISTING (36 sequences)

SEQ ID NO: 1 - DNA, 4206 bp, Wheat
atggagggcctcgcaagagagaccaacccatcatcccaccatcaagatttcaccgcctgtgcgagtgacgagcgcccggatgagtccgagttagaattggcatcgcgacagcgccagaatggtgctgcaaacaccgagcatgtgagtgagaacatgctgcttgacagcagcaagttgggagctctcaagaggcgcgagttcttcgacaacctgctaaagaacctcgaagacgaccacctccgctttctgcgcggacaaaaggaaagaattgacagggttgatgtcaagttgccagcaatagaggtgaggtataataatctgtttgtggaggcagagtgcagagttactaaaggaaatcacctgccatctctatggaatagtaccaaaggtgccttctcgggcctcgtgaagttgctaggcttcgaaacggaaagagcaaaaaccaacgttcttgaagatgtcagtggaatcatcaaaccctgcagattgactcttctactgggacctcctggatgtggcaaaagcactctgttgcgagctcttgccgggaaactagataaatctctaaaggtaacaggggatatctcttataatggttatgaacttcatgaatttgtacctgagaaaacagctgtgtatatcaaccaacatgatctgcacatagctgagatgactgtgagggaaactttagacttctcagcccagtgccaaggtgttggaagaagaccaaaaatactcaaggaggtgaacacaagggagagtgtggctgggatcatacctgatgcggacatcgatctatacatgaaggtagtagcagttgaagcttcagagcgaagcctacagacagattatattttgaagatcatggggctagagatatgcgcagacacgatggttggggatgcaatgagaagaggaatatcaggggggcagaagaaaagattaaccacagccgagatgattgtgggccccgcaagtgcatactttatggatgaaatatcaaatggtctggatagctctaccacttttcaaataatcaattgtttccagcaactgacaaacatcagcgagtacacgatggttatttcacttcttcaaccaacacctgaggtatttgatctttttgatgacctcatactaatggcagaagggaagattatctaccatggccctcgaaatgaagctctcaatttttttgaggagtgtgggttcatatgcccagaaagaaaagcggcagctgactttcttcaagagatcttgtcctggaaggaccaacaacagtactggttgggtccacatgaatcatacagatatatctcacctcatgaattatcaagcatgttcagggagaatcacagggggagaaaactacatgaacaaagtgtacctcccaaaagccagttgggcaaggaagctttagcattcaataagtattcgctacaaaaactggaaatgttcaaagcctgtggagcaagggaagcactcctaatgaaaaggaatatgtttgtttatgtcttcaaaacaggccagcttgccattattgcactcgtaacaatgtctgtattccttcgaactcgcatgacaataagtttcactcatgcaaattactatatgggagcattatttttttccatcatgattatgttaaatggcataccagagatgagcatgcagattgggagactcccaagtttttacaagcaaaagagctactatttctattcatcatgggcatatgcaataccagcttcagtcctaaaggtacctatttccatactggattcgcttgtatggatatctatcacatattatggtattggttatacacctactgtttcaaggttcttctgccagtttctgatactttgtcttctccatcattcagtcacctcgcagcatcgatttattgcttcatacttccaaacacctattgtgtctttcttctacctttttcttgctctaacagtattccttacattcggaggcttcattcttcccaagacctccatgccaggatggttaaactggggattttggatatctccaatgacatatgcagaaatcagcatagttattaacgaattcttggcaccaagatggcagaaggaaagtattcaaaacataacaattgggaaccaaatcctggttaatcatggcctatattacagttggcattattattggatatcctttggagccttgcttggatctattctcttattttatatcgcttttggattggcactagattacagaacacctacagaagaatatcatggaagcaggcctacaaagagcttatgtcaacagcaggaaaaagattacactattcaaaatgaatctgatgatcaatcaaatatttccaaagcaaaggtgactataccagttatgcatcttccaattacattccacaatctgaactactacattgataccccaccggaaatgctgaaacaaggctatccaacaagaagacttcgactgcttaataacataactggtgctttacgtcccggtgttctttctgcactaatgggtgttagtggagctgggaagacaactctactagatgtattagcaggaaggaaaacaggaggttatattgaaggggacataagaataggtggatatcccaaggtgcaggaaacatttgtcagaatcttgggttactgcgaacaagtcgacatacattccccacagcttacagttgaagagtctgtaacttattctgcgtggcttcgtctgccttctcatgtcgacgaacaaacaagatctaaatttgttgctgaagtccttgaaactgttgaactagatcaaataaaagatgtcttagtggggtcaccacagaaaaatggattgtccatggagcagagaaagaggctaacgattgcagtcgagcttgtttcaaacccatcaatcatactaatggatgaaccaacaacaggtttagatacaaggtcagcagccattgttattcgtgcagtcaaaaatatttgtgaaacaggaaggacagtagtctgtacaatccatcagccgagcactgaaatttttgaggcatttgatgagctcatattaatgaaaagcggtgggaaaacaatctacagtggaccaataggagagcgctcctgcaaagtgatcgagtactttgagaaaatttctggagtcccaaaaataaagagtaactgcaatccagctacttggatgatggatgtaacatcgacatcaatggaggttcaacacaatatggactttgcaattttgtatgaagagtcgtcactgcatagagaagctgaagatctagtggagcagctaagtatcccattaccaaattcagaaaatctatgtttctcccatagttttgcacagaatggctggattcaacttaaagcttgcttgtggaaacaaaacataacttactggaggagtcctcagtataacttgaggcgcattatgatgactgtcatatctgccctgatctacggaatattgttctggaagcatgcaaaagtattaaacaacgagcaggacatgctcagtgtttttggtgcaatgtatttgggtttcaccaccataggcgcttataatgatcagacaatcataccattcagtacaactgagcgtattgtaatgtatcgtgagagatttgcaggaatgtattcatcttggtcatattcattcgcacaggctttcattgagataccctatgtatttatccaagtggtactgtatacgttaattgtctatccgtcaactggttattattggacagcacacaaattcctatggttcttctacaccacattttgttcaattctctcctatgtttatgttgggttgcttcttgtttccataacccccaatgttcaagtagctaccatactggcttcatttttcaacaccatgcaaacactattctcaggatttattttacctgcacctcaaatcccgaagtggtggacttggctctactatctcactcctacatcttgggcactcaatgccctcttgacatcacaatacggaaacatagaaaaagaggtgaaagcatttggagaaactaaatcagtttcaatcttcttgaatgactattttgggtttcatcaagataagttgagcgtagtagcagctgtcctcgttgcctttccttttgtgttgataatcttgttttcgttgtccattgagaaacttaatttccagaagaggtaa

SEQ ID NO: 2 - protein, 1401 aa, Wheat
MEGLARETNPSSHHQDFTACASDERPDESELELASRQRQNGAANTEHVSENMLLDSSKLGALKRREFFDNLLKNLEDDHLRFLRGQKERIDRVDVKLPAIEVRYNNLFVEAECRVTKGNHLPSLWNSTKGAFSGLVKLLGFETERAKTNVLEDVSGIIKPCRLTLLLGPPGCGKSTLLRALAGKLDKSLKVTGDISYNGYELHEFVPEKTAVYINQHDLHIAEMTVRETLDFSAQCQGVGRRPKILKEVNTRESVAGIIPDADIDLYMKVVAVEASERSLQTDYILKIMGLEICADTMVGDAMRRGISGGQKKRLTTAEMIVGPASAYFMDEISNGLDSSTTFQIINCFQQLTNISEYTMVISLLQPTPEVFDLFDDLILMAEGKIIYHGPRNEALNFFEECGFICPERKAAADFLQEILSWKDQQQYWLGPHESYRYISPHELSSMFRENHRGRKLHEQSVPPKSQLGKEALAFNKYSLQKLEMFKACGAREALLMKRNMFVYVFKTGQLAIIALVTMSVFLRTRMTISFTHANYYMGALFFSIMIMLNGIPEMSMQIGRLPSFYKQKSYYFYSSWAYAIPASVLKVPISILDSLVWISITYYGIGYTPTVSRFFCQFLILCLLHHSVTSQHRFIASYFQTPIVSFFYLFLALTVFLTFGGFILPKTSMPGWLNWGFWISPMTYAEISIVINEFLAPRWQKESIQNITIGNQILVNHGLYYSWHYYWISFGALLGSILLFYIAFGLALDYRTPTEEYHGSRPTKSLCQQQEKDYTIQNESDDQSNISKAKVTIPVMHLPITFHNLNYYIDTPPEMLKQGYPTRRLRLLNNITGALRPGVLSALMGVSGAGKTTLLDVLAGRKTGGYIEGDIRIGGYPKVQETFVRILGYCEQVDIHSPQLTVEESVTYSAWLRLPSHVDEQTRSKFVAEVLETVELDQIKDVLVGSPQKNGLSMEQRKRLTIAVELVSNPSIILMDEPTTGLDTRSAAIVIRAVKNICETGRTVVCTIHQPSTEIFEAFDELILMKSGGKTIYSGPIGERSCKVIEYFEKISGVPKIKSNCNPATWMMDVTSTSMEVQHNMDFAILYEESSLHREAEDLVEQLSIPLPNSENLCFSHSFAQNGWIQLKACLWKQNITYWRSPQYNLRRIMMTVISALIYGILFWKHAKVLNNEQDMLSVFGAMYLGFTTIGAYNDQTIIPFSTTERIVMYRERFAGMYSSWSYSFAQAFIEIPYVFIQVVLYTLIVYPSTGYYWTAHKFLWFFYTTFCSILSYVYVGLLLVSITPNVQVATILASFFNTMQTLFSGFILPAPQIPKWWTWLYYLTPTSWALNALLTSQYGNIEKEVKAFGETKSVSIFLNDYFGFHQDKLSVVAAVLVAFPFVLIILFSLSIEKLNFQKR

SEQ ID NO: 3 - DNA, 20 bp, Artificial Sequence
  Synthetic oligonucleotide primer
tagcaaagggcgtcgattta

SEQ ID NO: 4 - DNA, 22 bp, Artificial Sequence
  Synthetic oligonucleotide primer
ctgttggaatgtcagaacttgc

SEQ ID NO: 5 - DNA, 23 bp, Artificial Sequence
  Synthetic oligonucleotide primer
agatgattgtgggccccgcaagt

SEQ ID NO: 6 - DNA, 25 bp, Artificial Sequence
  Synthetic oligonucleotide primer
ttacgagtgcaataatggcaagctg

SEQ ID NO: 7 - DNA, 31 bp, Artificial Sequence
  Synthetic oligonucleotide primer
ctatatgggagcattatttttttccatcatg

SEQ ID NO: 8 - DNA, 22 bp, Artificial Sequence
  Synthetic oligonucleotide primer
aaccatcctggcatggaggtct

SEQ ID NO: 9 - DNA, 36 bp, Artificial Sequence
  Synthetic oligonucleotide primer
cggaattcgattacagaacacctacagaagaatatc

SEQ ID NO: 10 - DNA, 35 bp, Artificial Sequence
  Synthetic oligonucleotide primer
cgggatccttacctcttctggaaattaagtttctc

SEQ ID NO: 11 - DNA, 53 bp, Artificial Sequence
  Synthetic oligonucleotide primer
ggggacaagtttgtacaaaaaagcaggcttcatggagggcctcgcaagagaga

SEQ ID NO: 12 - DNA, 54 bp, Artificial Sequence
  Synthetic oligonucleotide primer
ggggaccactttgtacaagaaagctgggtcctggcctgttttgaagacataaac

SEQ ID NO: 13 - DNA, 58 bp, Artificial Sequence
  Synthetic oligonucleotide primer
ggggacaagtttgtacaaaaaagcaggcttcatggattacagaacacctacagaagaa

SEQ ID NO: 14 - DNA, 54 bp, Artificial Sequence
  Synthetic oligonucleotide primer
ggggaccactttgtacaagaaagctgggtccctcttctggaaattaagtttctc

SEQ ID NO: 15 - DNA, 54 bp, Artificial Sequence
  Synthetic oligonucleotide primer
ggggacaagtttgtacaaaaaagcaggcttcatggcagaaaccattgatctaac

SEQ ID NO: 16 - DNA, 53 bp, Artificial Sequence
  Synthetic oligonucleotide primer
ggggaccactttgtacaagaaagctgggtctttgccctttccttttcccttgc

SEQ ID NO: 17 - DNA, 29 bp, Artificial Sequence
  Synthetic oligonucleotide primer
cgggatccatggagggcctcgcaagagag

SEQ ID NO: 18 - DNA, 29 bp, Artificial Sequence
  Synthetic oligonucleotide primer
cgggatccttacctcttctggaaattaag

SEQ ID NO: 19 - DNA, 22 bp, Artificial Sequence
  Synthetic oligonucleotide primer
agctgactttcttcaagagatc

SEQ ID NO: 20 - DNA, 26 bp, Artificial Sequence
  Synthetic oligonucleotide primer
tttttgtagcgaatacttattgaatg

SEQ ID NO: 21 - DNA, 21 bp, Artificial Sequence
  Synthetic oligonucleotide primer
cttcagtcctaaaggtcccta

SEQ ID NO: 22 - DNA, 21 bp, Artificial Sequence
  Synthetic oligonucleotide primer
gaaactggcagaagaaccttg

SEQ ID NO: 23 - DNA, 21 bp, Artificial Sequence
  Synthetic oligonucleotide primer
atggaagctgctggaatccat

SEQ ID NO: 24 - DNA, 24 bp, Artificial Sequence
  Synthetic oligonucleotide primer
ccttgctcatacggtcagcaatac

SEQ ID NO: 25 - DNA, 22 bp, Artificial Sequence
  Synthetic oligonucleotide primer
agaatggtgctgcaaacaccgg

SEQ ID NO: 26 - DNA, 30 bp, Artificial Sequence
  Synthetic oligonucleotide primer
gtgtcttatcagttccattctcattgattc

SEQ ID NO: 27 - DNA, 20 bp, Artificial Sequence
  Synthetic oligonucleotide primer
gcaaagggcgtcgatttaag

SEQ ID NO: 28 - DNA, 26 bp, Artificial Sequence
  Synthetic oligonucleotide primer
atcagttccattttcattgagttggt

SEQ ID NO: 29 - DNA, 17 bp, Artificial Sequence
  Synthetic oligonucleotide primer
cgccgccacggccctcc

SEQ ID NO: 30 - DNA, 34 bp, Artificial Sequence
  Synthetic oligonucleotide primer
ctacatcccataatgtaaaatgttttttgatgag

SEQ ID NO: 31 - DNA, 23 bp, Artificial Sequence
  Synthetic oligonucleotide primer
atgtaggtctgcctagttgggcg

SEQ ID NO: 32 - DNA, 29 bp, Artificial Sequence
  Synthetic oligonucleotide primer
gctttaatgacagctccctggccaacttc

SEQ ID NO: 33 - DNA, 17 bp, Artificial Sequence
  Synthetic oligonucleotide primer
cgccgccacggccctcc

SEQ ID NO: 34 - DNA, 33 bp, Artificial Sequence
  Synthetic oligonucleotide primer
ctacatcccataatgtaaaatgttttttgatga

SEQ ID NO: 35 - DNA, 23 bp, Artificial Sequence
  Synthetic oligonucleotide primer
atgtaggtctgcctagttgggcg

SEQ ID NO: 36 - DNA, 29 bp, Artificial Sequence
  Synthetic oligonucleotide primer
gctttaatgacagctccctggccaacttc

(19) United States
(12) Patent Application Publication
(10) Pub. No.: US 2014/0359898 A1
(43) Pub. Date: Dec. 4, 2014

(54) TRANSGENIC CEREAL PLANTS WITH INCREASED RESISTANCE TO RUST DISEASES

(71) Applicant: THE BOARD OF REGENTS FOR OKLAHOMA STATE UNIVERSITY, STILLWATER, OK (US)

(72) Inventors: Liuling Yan, Stillwater, OK (US); Brett Carver, STILLWATER, OK (US); Tilin Fang, STILLWATER, OK (US); Robert M. Hunger, STILLWATER, OK (US)

(73) Assignee: THE BOARD OF REGENTS FOR OKLAHOMA STATE UNIVERSITY, STILLWATER, OK (US)

(21) Appl. No.: 14/294,904

(22) Filed: Jun. 3, 2014

Related U.S. Application Data
(60) Provisional application No. 61/830,444, filed on Jun. 3, 2013.

(51) Int. Cl. C12N 15/82

(52) U.S. Cl. CPC C12N 15/8282
USPC 800/279; 800/301

(57) ABSTRACT

A transgenic wheat 2174 cultivar with increased resistance to diseases caused by foliar pathogens is provided, as are methods for making the transgenic cultivar. The methods involve genetically engineering (transforming) 2174 to overexpress cDNA encoding the resistance gene LR34 in a form that is correctly spliced.

### CROSS REFERENCE TO RELATED APPLICATIONS

This application claims the benefit of U.S. Provisional Patent Application Ser. No. 61/830,444 filed on Jun. 3, 2013, and incorporates said provisional application by reference into this document as if fully set out at this point.

### STATEMENT REGARDING FEDERAL SPONSORED RESEARCH OR DEVELOPMENT

This invention was made with U.S. Government support under USDA/NIFA Grant No. 2011-68002-30029 awarded by the Department of Agriculture. The Government has certain rights in this invention.

### FIELD OF THE INVENTION

The present invention relates generally to the field of improved crop plants in general and, more specifically, to a transgenic 2174 wheat cultivar with increased resistance to rust diseases.

### BACKGROUND

Wheat is a major food crop worldwide, but is constantly challenged by several fungal diseases, such as leaf rust and stripe rust [1,2]. There are numerous genes or quantitative trait loci (QTLs) that have been mapped for host plant resistance to epidemic pathogens, including 67 genes/QTLs for stripe rust and 49 for leaf rust [3]. The resistance of these genes/QTLs can be divided into two types: i) complete or qualitative resistance, and ii) partial or quantitative resistance [4-11]. Qualitative resistance fits a gene-for-gene model, in which a pathogen is recognized by a plant host resistance gene resulting in complete exclusion of the pathogen [12]. Quantitative resistance remains poorly understood but is explained by two hypotheses: i) a consequence of interactions among multiple resistance genes [13,14], or ii) the outcome of direct interactions between pathogen effectors and plant defense proteins or indirect interactions between pathogen effectors and plant defense proteins triggered by the effectors [15,16].

Lr34 is a non-race-specific gene that confers resistance against fungal pathogens causing leaf rust, stripe rust, and powdery mildew, but also against barley yellow dwarf virus [17-20]. Because of the durability and non-specificity of its resistance against multiple pathogens, Lr34 has become one of the most important disease-resistance genes in wheat worldwide and has been utilized since the early 21st century [21-23]. Cloning of Lr34 indicated that the resistant allele of the gene consists of 24 exons and 23 introns spanning 11,805 by nucleotide sequence from the start codon to the stop codon, and it encodes a pleiotropic drug resistance (PDR)-like adenosine triphosphate-binding cassette (ABC) transporter, which consists of two units each containing a cytosolic nucleotide binding domain (NBD) and a hydrophobic transmembrane domain (TD) [2]. The resistant allele Lr34r and the susceptible allele Lr34s can be distinguished by three polymorphisms. The first is that the codon TTC encoding phenylalanine is present in exon 11 of the Lr34E11s allele but absent in the Lr34E11r allele, and the second is that a point mutation in exon 12 from ‘C’ to ‘T’ produces a tyrosine residue in the Lr34E12s protein and a histidine residue in the Lr34E12r protein [2,18]. The third is that Lr34E22s contains a point mutation in exon 22 that results in a premature stop codon and a shortened, non-functional Lr34E22s protein lacking 185 amino acids covering the majority of the second transmembrane domain [2, 18, 22, 24]. Each of the three mutations has been characterized at the protein level but not at the level of transcription.

Previous studies on winter wheat cultivars adapted to the southern Great Plains indicated that Lr34 accounted for 18 to 35% of the total phenotypic variation in leaf rust reaction among recombinant inbred lines (RILs) from the biparental cross of ‘Jagger’ carrying a susceptible Lr34 allele and ‘2174’ carrying a resistant Lr34 allele [22]. Lr34 was also reported to account for 29 to 33% of the variance for leaf rust reaction in other studies [2, 13, 25]. However, the molecular mechanism for the partial resistance conferred by this gene remains largely unknown. As a result, the deployment of the Lr34 gene in wheat breeding is still based on the conventional breeding approach, by which a resistant allele from a donor is introduced to a recipient cultivar carrying a susceptible allele.

A thorough understanding of Lr34 transcriptional defects and its effects on further downstream or upstream proteins would contribute significantly to the establishment of durable disease resistance pathways, and permit genetic engineering of wheat that is resistant to rust and other fungal diseases.

### SUMMARY OF THE INVENTION

Lr34 is a non-race-specific gene that confers resistance against fungal pathogens including leaf rust, stripe rust, stem rust, and powdery mildew, as well as barley yellow dwarf virus. Because of the durability and non-specificity of its resistance against multiple pathogens, Lr34 has become one of the most important disease-resistance genes in wheat worldwide. However, the resistance of Lr34 is partial and it is thus called a ‘minor’ gene.

The present disclosure describes the novel mechanism that underlies the partial resistance against leaf rust by Lr34 in adult plants of wheat (Triticum aestivum, 2n=6x=42, AABBDD). As explained more fully below, in a cultivar carrying the resistant Lr34 allele, only a portion (35%) of its transcripts were correctly spliced and the majority (65%) of its transcripts were incorrectly spliced due to multiple mis-splicing events. The Lr34 splicing was sensitive to low temperature, which produced a higher proportion and more forms of mis-spliced transcripts in seedlings, compared with adult plants. The proteins deduced from the mis-spliced cDNAs were incomplete in length and void of an interaction site for TaFKBP1 (the first characterized immunophilin protein in T. aestivum).

However, when a correctly spliced Lr34 cDNA was over-expressed in a transgenic cultivar that carries the resistant allele, functional Lr34 transcript levels were significantly increased and the transgenic wheat displayed increased resistance to leaf rust. These findings show that the level of resistance conferred by a quantitative gene can be increased by overcoming the effects of mis-splicing.

### BRIEF DESCRIPTION OF THE DRAWINGS

FIG. 1A-K. Structure of mis-spliced cDNAs derived from Lr34. A, The Lr34 gene structure consisting of 24 exons and 23 introns. B, The correctly spliced Lr34 mRNA. C-I, the left panels: locations of alternative splicing events described as skipped or cassette exon (CE), intron retention (IR), alternative 3′ splicing sites (A3SS), and alternative 5′ splicing sites (A5SS). C, Partial exon 10 skipping, 92 by (C1) and 11 by (C2) at the 5′ end of exon 10. D, 44 by at the 5′ end of exon 12 skipping. E, Complete exon 16 skipping. F, Complete intron 1 retention; G, Complete intron 9 retention. H, Complete intron 14 retention. I, Intron 6 was partially retained with 12 by (G1) or 17 by (G2) at the 3′ end of this intron respectively. C-I, the right panels, deduced proteins of Lr34. Grey boxes indicate exons, lines indicate introns, dotted line boxes indicate the skipped exons, black boxes indicate the retained introns, stars indicate mis-spliced fragments in mRNA, and hearts indicate the prematured stop codon. J, The non-functional Lr34 of Jagger. K, Protein structure of functional Lr34 of 2174, consisting of two units, each of which contains a cytosolic nucleotide binding domain (NBD) and a hydrophobic transmembrane domain (TD).

FIG. 2A-C. Subcellular localization and in vivo interaction of transiently expressed Lr34 and TaFKBP1 proteins in living cells. A, The subcellular location of TaFKBP1-YFP protein expressed by pEG101 on the cytoplasma membranes (M) as well as the nucleus (N). The images were taken with a bright filter (BF) to indicate the background of the leaves infiltrated with A. tumefaciens carrying constructs, or with an ultraviolet filter (DAPI) to indicate the position of the nucleus stained with 4′,6-diamidino-2-phenylindole. The overlay images align the locations of GFP with the DAPI-stained nucleus. B, The subcellular locations of two Lr34-YFP protein fragments {(amino acids from position 1 to 510 and from position 750 to 1401(end)} expressed by pEG101 in the cytoplasma membranes of living cells in tobacco leaves. C, BiFC analysis of Lr34 and TaFKBP1 proteins. The yellow fluorescent proteins resulted from the in vivo interaction between Lr34-YN and TaFKBP1-YC. An image was taken under a fluorescent microscope with GFP filter to indicate the absence of signal when Lr34 and YC vector interaction or TaFKBP1 and YN vector interaction was used for negative control. The scale bar in all images represents 50 μm.

FIG. 3A-D. Genetic effects of TaFKBP1 on resistance to diseases. A, PCR marker for TaFKBP-A1. The PCR products were directly run in a 1% agarose gel, showing polymorphic bands from the Jagger allele, but not from the 2174 allele. Chinese spring nulli-tetrasomic lines were used to determine the chromosomal location of TaFKBP-A1. CS2: N1AT1D; CS4: N1BT1D; CS6: N1DT1B. B, PCR marker for TaFKBP-B1. The PCR products digested with restriction enzyme DdeI were run in a 1% agarose gel. 1. Undigested PCR products of Jagger, 2. Undigested PCR products of 2174. 3. Digested PCR products of Jagger (750 bp), 4. Digested PCR products of 2174 (448 bp). C, TaFKBP-A1 effect on stripe rust. D, TaFKBP-B1 effect on stripe rust. The phenotype was averaged from each line of the population (n=96) that was tested on stripe rust at on Washington State for two locations/years (Fang et al., 2011). The significant effects of the two TaFKBP1 genes on resistance against these diseases were determined using one-way analysis of variance (ANOVA). Bar indicates standard error. Y axis indicates the rate of resistance against stripe rust.

FIG. 4A-D. Transcript levels and phenotypes of over-expressed Lr34 in transgenic wheat. Transgenic plants of winter variety 2174 transformed with Lr34 cDNA fused with pHAC20 vector. A, Transcript levels of Lr34 in leaves from adult plants using primers to amplify the region covering exon 10. Y axis indicates the values calculated by the 2^{(-ΔΔCT)} method, where CT is the threshold cycle, and actin was used as an endogenous control. B, Transcript levels of Lr34 in leaves from adult plants using primers to amplify the region covering exon 12. C, Number of pustules counted on positive plants compared to wild type plants. D, Percentage of leaf area covered with leaf rust pustules compared between positive plants and wild type plants. Values are averages of 16 to 20 plants, and bar indicates standard error (±SE).

FIGS. 5A and B. Comparison of patterns of mis-spliced transcripts among RILs. A, Frequency distribution of the 60 recombinant inbred lines (RILs) carrying the resistant Lr34 allele selected from the Jagger×2174 RIL population for percent infection score averaged across three environments/years. B, Expression of normal and mis-spliced Lr34 using primers Lr34-DMS-F2 and Lr34-DMS-R2 to amplify regions covering exon 10 (E10) and primers Lr34-DMS-F2 and Lr34-DMS-R2 exon 12 (E12). The specificity of the primers to Lr34 was determined, by comparison with Lr34-B on homoeologous genome 4A (see FIG. 9B) and Lr34-A on homoeologous genome 7A (see FIG. 9A) that were sequenced and mapped in this study. Those primers specific to Lr34 were not able to amplify gDNAs, if any was contaminated in the cDNA samples, because one of each pair covered two exons. For partial E10 skipping, the PCR product size from normal Lr34 transcripts was 597 bp, and partial E10 skipping fragment was 505 bp. For partial E12 skipping, the normal Lr34 transcript was 414 bp, and the fragment with skipped partial exon 12 was 370 bp. M: DNA ladder marker; Lane 1-9: 9 lines that have the resistant Lr34 allele in the RILs population. Lane 10: mis-spliced cDNA (PCR products from the plasmid containing mis-spliced Lr34 cDNA). Lane 11: normal cDNA (PCR products from the plasmid containing normal Lr34 cDNA.

FIGS. 6A and B. Mis-spliced Lr34 transcripts in wheat cultivars. A, Mis-spliced Lr34 transcripts using primers Lr34-DMS-F2 and Lr34-DMS-R2 to amplify regions covering exon 10. B, Mis-spliced Lr34 transcripts using primers Lr34-DMS-F3 and Lr34-DMS-R3 to amplify regions covering exon 12. M. Marker; 1. Jagger; 2. 2174; 3. OK bullet; 4. Duster; 5. Billings; 6. Endurance; C1. clone 1 that has intron 9 retention; C2. clone 2 that has partial exon 10 (92 bp) skipped; C2. clone 2 that has partial exon 12 (44 bp) skipped; C0, clone that has correct spliced exons and introns. The samples of these cultivars were collected from adult plants in field trials in 2010 at Stillwater, Okla.

FIG. 7A-C. Alternative sites of intron 6 in Lr34. A, Intron 6 was correctly spliced out and exon 6 and exon 7 were joined. B, The underlined 12 by at the 3′ end of intron 6 were retained. C, The underlined 17 by at the 3′ end of intron 6 were retained.

FIGS. 8A and B. Chromosomal locations of TaFKBP-A1 and TaFKBP-B1. A, TaFKBP-A1 on the long arm of chromosome 1A. B, TaFKBP-B1 on the long arm of chromosome 1B.

FIG. 9A-C Genetic linkage maps of Lr34-A, Lr34-B, and Lr34 genes. The chromosomal locations of Lr34-A and Lr34-B were reported [26], but their sequences are not released in GenBank. Each of the two alleles for each of two homoeologous genes was sequenced, Lr34-A for the Jagger allele and the 2174 allele, Lr34-B for the Jagger allele and the 2174 allele. A, Lr34-A was mapped on chromosome 7A. B, Lr34-B was mapped on chromosome 4A representing a translocated fragment from chromosome 7B. Blue bar indicates the translocated fragment from chromosome 7B. C, Lr34 was mapped on chromosome 7D [2]. The Lr34-A gene was mapped by using primer Lr34-X-F2 to combine with primer Lr34-In2-R1 for PCR under the following conditions: denature at 94° C. for 3 min, amplification for 40 cycles with 94° C. for 30 s, at 55° C. for 30 s, and 72° C. for 1 min per cycle, and final extension at 72° C. for 10 min. The PCR products were characterized by electrophoresis on the 1% agarose gel. For Lr34-A, the 2174 allele showed a 734 by band and the Jagger allele showed a 439 by band. Only one polymorphism was found in the 12 kb Lr34-B gene between Jagger and 2174 alleles—one “T” indel in intron 1 an 8 “T” repeat in Jagger and 9 “T” repeat in 2174. Lr34-B was mapped by sequencing each line of the population using primers Lr34B-5-F1 and Lr34-In2R1 and PCR conditions: denature at 94° C. for 3 min, amplification for 40 cycles with 94° C. for 30 s, at 58° C. for 30 s, and 72° C. for 1 min per cycle, and final extension at 72° C. for 10 min). Then the PCR products were purified with Wizard SV Gel and PCR Clean-Up System (by Promega, Madison, Wis., USA), and the purified PCR products were directly sequenced. The genetic maps were constructed in the Jagger×2174 population of 96 RILs.

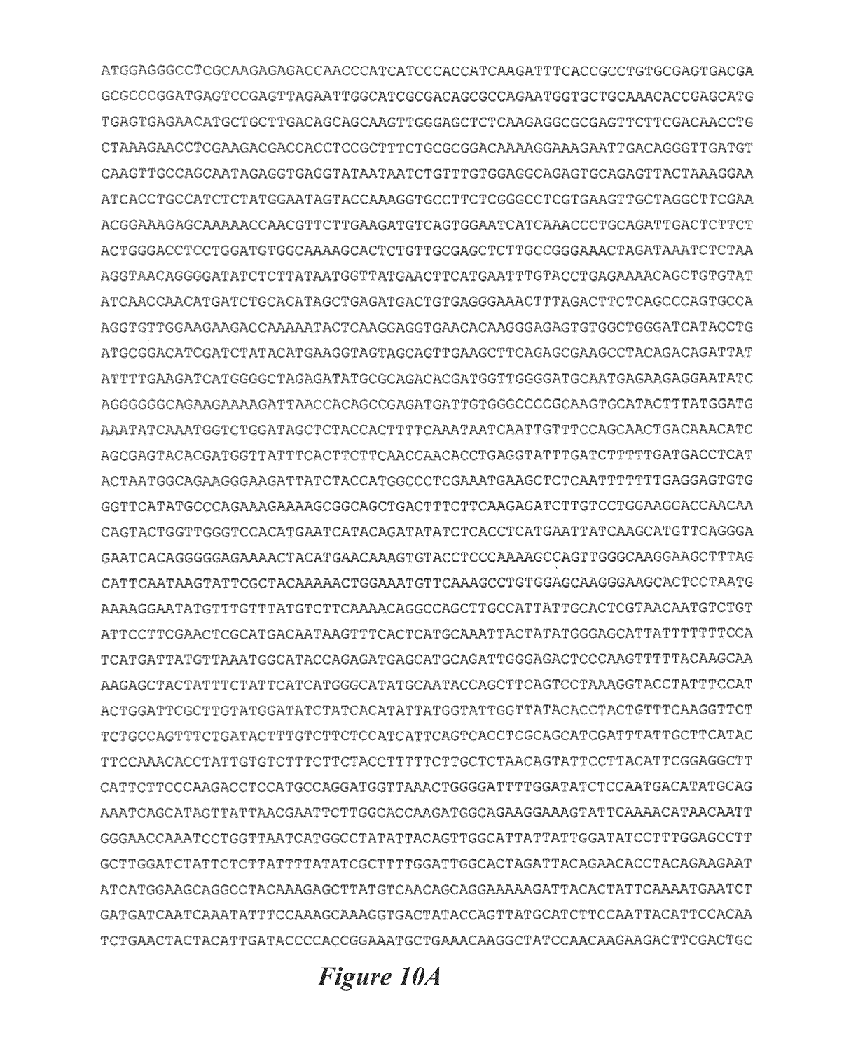

FIGS. 10A and B. The nucleotide sequence of Lr34 cDNA that was used for transformation. Start and stop codons are shaded.

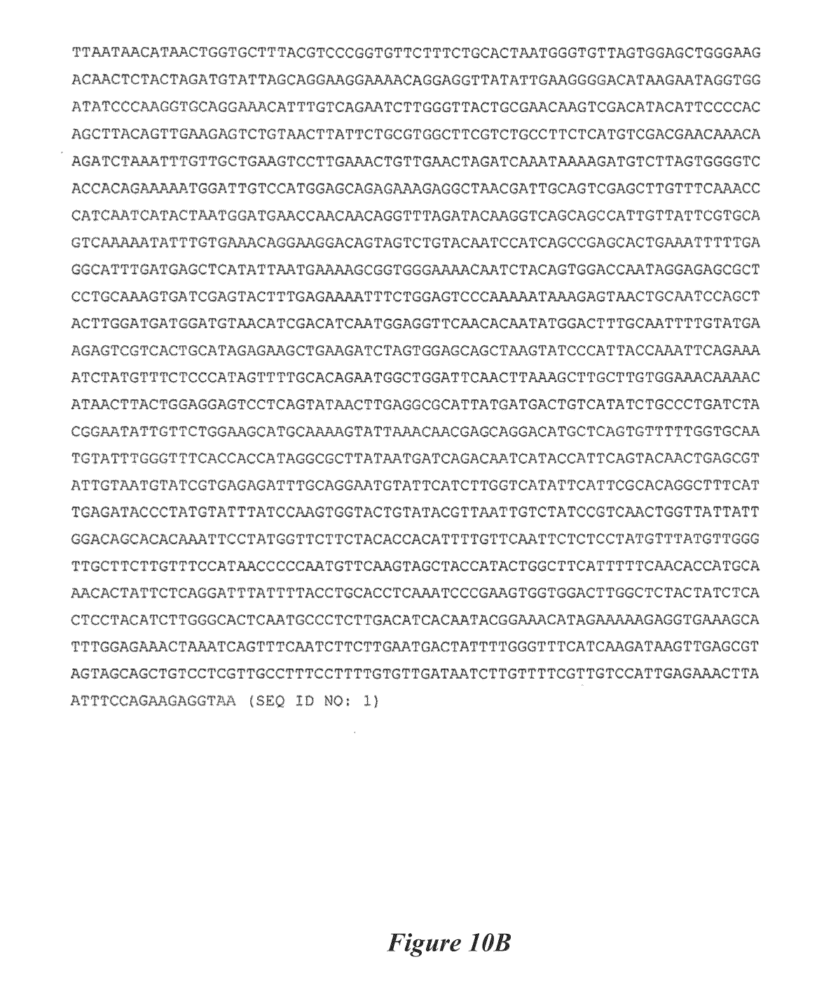

FIG. 11. The translated protein sequence of Lr34 cDNA that was used for transformation (* indicates stop)

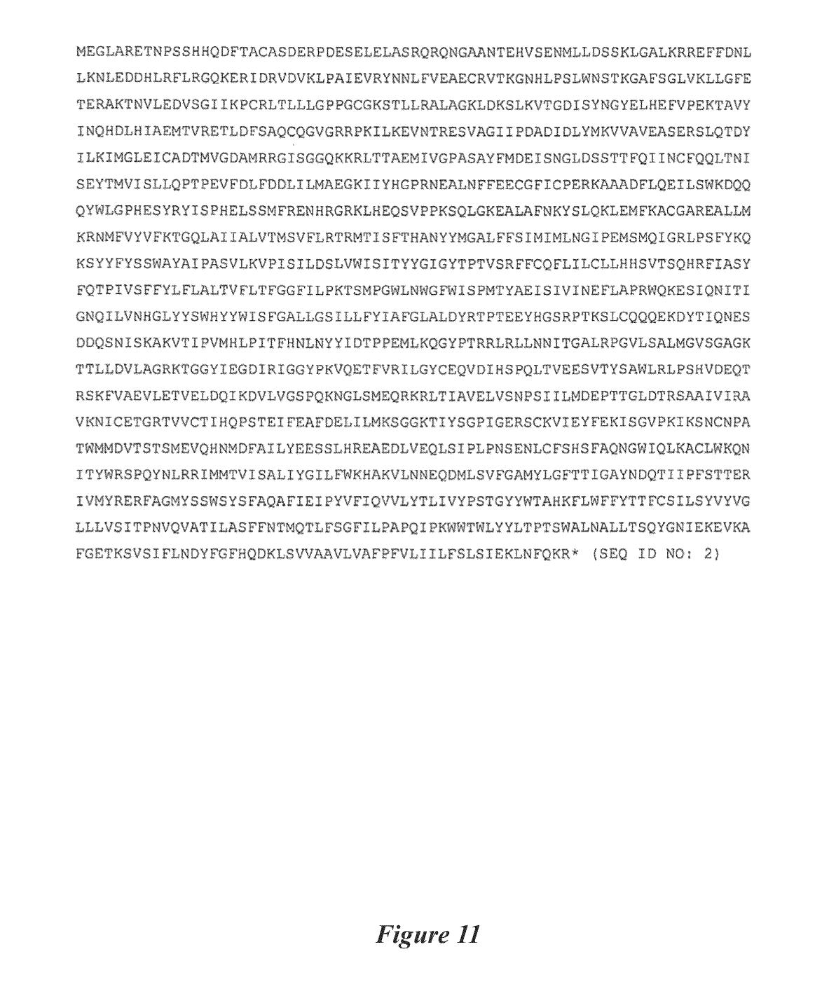

### DETAILED DESCRIPTION

As described in the Examples section below, it has been discovered that, in 2174 wheat cultivar which has a native or wild-type rust resistant Lr34 gene, only a portion of the Lr34 gene RNA transcripts are correctly spliced and the majority of transcripts are incorrectly produced. Thus, even though a cultivar has the resistance gene, the gene does not fully exert its phenotypic role because a large percentage of its gene products are not functional. This mechanism is likely responsible for the partial resistance of minor or quantitative genes in other plants and organisms as well. The invention accordingly provides methods of increasing disease resistance in a wheat cultivar which contains a native Lr34 resistance gene (e.g. the 2174 cultivar) by genetically engineering (transforming) the cultivar or cell or the cultivar to overexpress a full-length, correctly spliced nucleic acid sequence that encodes a full-length Lr34 resistance protein (e.g. a cDNA sequence based on a correctly spliced mature mRNA sequence, which lacks introns but contains Lr34 encoding exons). Provision of the full-length gene, without introns, obviates or eliminates the need for splicing of pre-mRNA, since “mature” mRNA encoding the resistance gene is produced directly from the gene sequence. In other words, pre-mRNA processing to remove introns and join exons is not necessary. Instead, mRNA is transcribed from the encoding DNA directly into “processed” mRNA, and a viable, active gene product is translated from the mRNA. It is noted that in this aspect of the invention, naturally occurring resistance gene transcripts may still be (and likely are) produced from extant (non-recombinant) native DNA in the cell, and a fraction of those native transcripts may be spliced correctly, so that the level of active protein that is produced is the sum of that which is produced as a result of genetic transformation of the plant, plus the fraction (if any) of active protein that is correctly produced from native, unmodified gene sequences.

According to the invention, the gene that is overexpressed in a transgenic 2174 wheat plant is a recombinant Lr34 gene (e.g. a cDNA) which does not encode introns, as set forth in SEQ ID NO: 1. The gene may be referred to as a “transgene” i.e. it is a recombinant, non-native gene sequence. The primary amino acid sequence of recombinant Lr34 produced from this cDNA is presented in SEQ ID NO: 2. Those of skill in the art will recognize that an active Lr34 gene product (e.g. a protein that is functional and which has resistance activity) may be encoded by SEQ ID NO: 1 or by other similar sequences which encode either the same polypeptide sequence, e.g. using alternative codons (due to redundancy of the genetic code), or which encode a different polypeptide sequence with the desired resistance activity. Generally, at least about 25, 30, 40, 45, 50, 55, 60, 65, 70, 75, 80, 85, 90, 95 or more of the resistance activity is retained in such variant sequences.

Variant polypeptide sequences may, for example, contain conservative or non-conservative amino acid substitutions, or may be truncated at the carboxyl and/or amino termini, or may have various deletions or additions within the polypeptide chain, so long as the changes are tolerated by the transcribed protein and a sufficient or desired or useful level of activity is exhibited by the variant protein. Further, modified, variant polypeptide sequences may contain additional sequences such as leader sequences, or detectable sequences, or sequence which facilitate genetic manipulation (e.g. sequences which facilitate cloning of the encoding DNA into a vector), etc. Generally, the encoded Lr34 will have a level of amino acid identity (identical amino acids at identical positions, or if gaps or insertions are introduced, at corresponding positions, as calculated by known matrices) that is at least about 50, 55, 60, 65, 70, 75, 80, 85, 90, 95, 96, 97, 98, 99 or 100% that of SEQ ID NO: 2; or will have a level of amino acid similarity (similar conservative equivalent amino acids at the same positions or if gaps or insertions are introduced, at corresponding positions, as calculated by known matrices) that is at least about 50, 55, 60, 65, 70, 75, 80, 85, 90, 95, 96, 97, 98, 99 or 100% of that of SEQ ID NO: 2 All such Lr34 derived or related sequences are encompassed herein, as are the nucleic acids encoding them (e.g. DNA, cDNA, RNA, mRNA, DNA and RNA hybrids, etc., both single and double stranded).

Transgenic plants and plant cells (i.e. genetically modified or transformed plants/cells) which have been genetically engineered or transformed in one or both of these manners are also provided, as are products which are made of or made from such transgenic plants and cells, and progeny thereof. As used herein, “plant” and “plant cell” encompasses any and all parts of a plant and individual plants cells or plant tissue from any and all parts of a plant, including reproductive organs or structures, or portions thereof (e.g. slices, individual blades, kernels, fibers isolated therefrom, etc.), whether alive or non-living For example, included are stems, leaves, flowers, roots, seeds, pollen, blades, “heads”, fruits, embryos, isolated cells (e.g. cells isolated from a plant or cells grown and/or maintained in vitro, e.g. in a laboratory, or clones thereof), straw, etc. Plants may be “in the field”, or already harvested, or harvested and processed (e.g. dried and/or separated into component parts, etc.).

In some aspects, in the transgenic 2174 cultivars of the invention the level of resistance conferred by transformation with lr34 cDNA is increased to the level that would be conferred by a qualitative gene, i.e. the level of resistance to the disease of interest is substantially complete. However, those of skill in the art will recognize that much benefit accrues even if resistance to an infectious pathogen is not complete (100%) but is in the range of an increase in resistance of at least about 10, 15, 20, 25, 30, 35, 40, 45, 50, 55, 60, 65, 70, 75, 80, 85, 90 or 95%. Such percentages are determined or calculated, for example, in comparison to a corresponding control plant/plant cell that has not been transformed to overexpress a full-length, correctly spliced nucleic acid sequence that encodes the resistance gene Lr34. The control plant or plant cell may be a natural plant/plant cell that contains one or more genes encoding Lr34 in its native, naturally occurring state, or may be a transgenic plant or plant cell that has already been genetically engineered to contain other gene sequences of interest, e.g. other than resistance gene encoding sequences, genes that confer resistance to pesticides, etc. Those of skill in the art are familiar with assessing levels of disease resistance.

The genetically engineered cultivars of the invention are generated in a laboratory by altering their genetic makeup. This is usually done by adding one or more genes to a plant's genome using genetic engineering techniques. Exemplary methods of genetically engineering plants include but are not limited to: the biolistic method (particle gun), by using Agrobacterium tumefaciens mediated transformation, nanoparticles, etc. Any suitable technique may be used to transform the 2174 wheat cultivars

Plant transformation generally involves the construction of an expression vector which will function in plant cells. Such a vector comprises DNA comprising a gene under control of, or operatively linked to, a regulatory element (for example, a promoter), i.e. expression of genes included in expression vectors is driven by a nucleotide sequence such as a promoter. As used herein, “promoter” refers to a region of DNA upstream from the start of transcription and involved in recognition and binding of RNA polymerase and other proteins to initiate transcription. A “plant promoter” is a promoter capable of initiating transcription in plant cells. The expression vector may contain one or more such operably linked gene/regulatory element combinations. The vector(s) may be in the form of a plasmid and can be used alone or in combination with other plasmids to provide transformed plants using transformation methods known in the art and/or described herein to incorporate transgenes into the genetic material of the plant(s) or plant cells. In one aspect, the invention encompasses a vector comprising a nucleic acid sequence encoding an Lr34 resistance gene, as described herein.

Several types of promoters are well known in the transformation arts as are other regulatory elements that can be used alone or in combination with promoters. Exemplary promoters include but are not limited to: those described herein (e.g. the promoter of vector pHAC20; promoters described in U.S. Pat. No. 8,729,355, the complete contents of which are hereby incorporated by reference in entirety; the soybean elongation factor 1 (EF1) promoter described in U.S. Pat. No. 8,710,206, the complete contents of which are hereby incorporated by reference in entirety; etc. The promoters may be constitutive or inducible, and may function in all parts of the plant or may be tissue selective or tissue-preferred or tissue- or cell-specific.

Expression or overexpression of a gene in a host organism such as a plant or plant cell involves transcription of the gene and translation of the mRNA into a precursor or mature protein. As used herein, “overexpression” refers to the production of a gene product in transgenic organisms that exceeds levels of production in normal, native, wild-type or non-transformed control plants. Transgenic plants may be referred to as “cisgenic plants” if they are made using genes found within the same species or a closely related species.

Resistance to numerous diseases may be increased by the practice of the methods described herein. Exemplary diseases include but are not limited to foliar diseases such as leaf rust, stripe rust, powdery mildew, and barley yellow dwarf virus.

The transgenic 2174 wheat cultivar described herein may be used for any purpose, e.g. as food (as grain, flour, sprouted grain, shoots or rhizomes); in fermented products such as alcoholic drinks; as pasture or hay for livestock; in the manufacture of various wheat-based products; or even as landscaping or ornamental plants grasses, or for any other suitable purpose. In addition to the transgenic plants themselves, products made from or with the transgenic plants are also encompassed by the invention.

### EXAMPLES

In the present disclosure it is reported that the partial resistance of an endogenous Lr34 occurs because the majority of its transcripts are incorrectly spliced due to intron retention or exon skipping. This finding leads to a novel strategy of breeding cultivars resistant to rust pathogens, in which regulators that result in mis-spliced transcripts of Lr34 are mutated to increase endogenous Lr34 transcript level and thus resistance to multiple diseases in wheat.

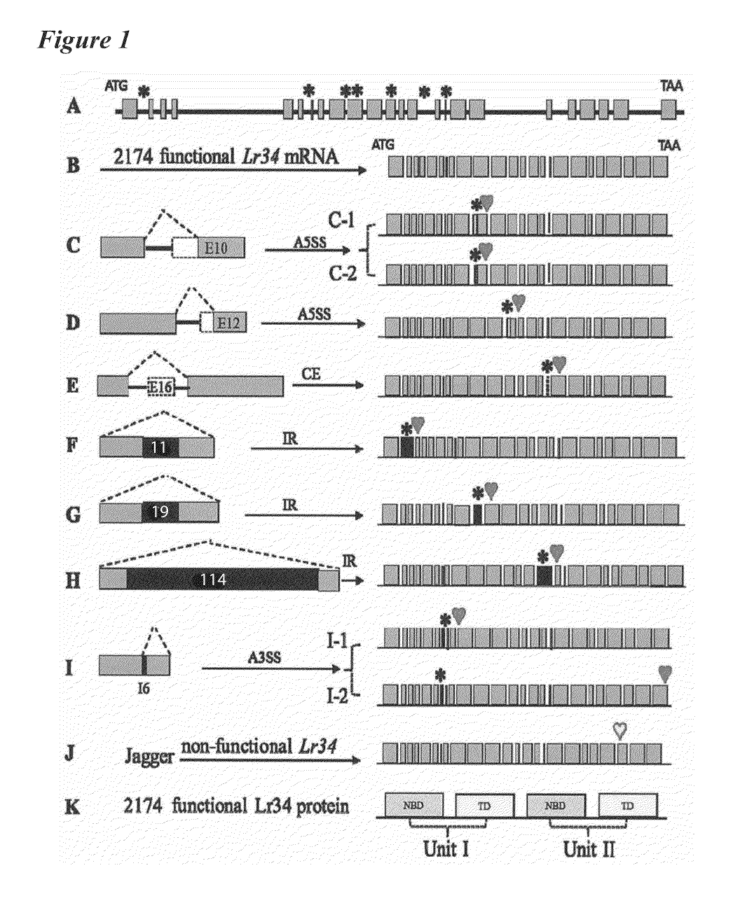

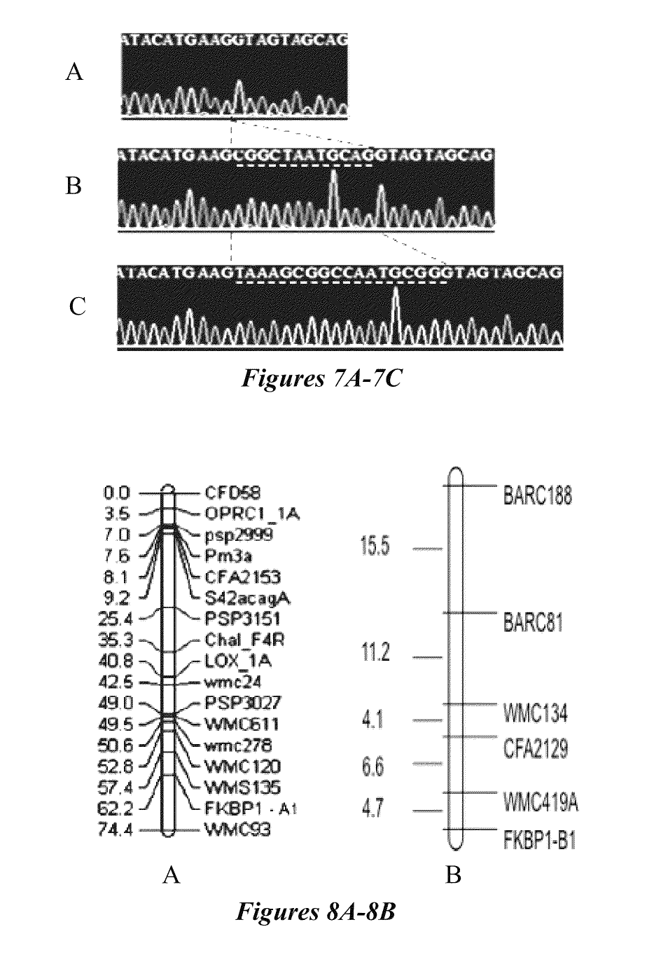

In one embodiment, as a first step to use Lr34 to create transgenic wheat to address this mechanistic issue, we attempted to amplify a complete cDNA of Lr34 from 2174. Lr34 consists of 24 exons and 23 introns (FIG. 1A), and a resistant Lr34 allele in 2174 should be functional if all of its introns and exons were correctly spliced (FIG. 1B). Surprisingly, however, a total of 23 Lr34 cDNA clones were completely sequenced, and 15 (65%) cDNAs were found incorrectly spliced (Table 1). Nine unique mis-splicing events were observed, including four involving exon skipping and five involving intron retention. The exon skipping events included two partial skipping at the same exon: 92 by (FIG. 1C1) or 11 bp (FIG. 1C2) at the 5′ end of exon 10, a partial skipping of 44 by at the 5′ end of exon 12 (FIG. 1D), and a complete exon skipping of exon 16 (FIG. 1E). The intron retention events included three introns that were completely retained: intron 1 (99 bp) (FIG. 1F), intron 9 (92 bp) (FIG. 1G), and intron 14 (437 bp) (FIG. 1H), and intron 6 that was partially retained with 12 by or 17 by at the 3′ end of this intron (FIG. 1I, FIG. 7). Since a mis-spliced transcript with low expression level could not be sampled in cDNA clones sequenced, it is anticipated that many more splicing events exist in Lr34 transcripts.

TABLE 1
Various mis-splicing events in Lr34 transcripts.

| Clone | | | Size |
|---|---|---|---|
| code # | Mis-splicing type | Mis-splicing site | (bp) |
| LY2879 | Intron retention | Complete intron 1 | 99 |
| LY2637 | Exon skipping | Partial exon 10 at 5′ end | 92 |
| LY2638 | Intron retention | Complete intron 9 retention | 92 |
| | Exon skipping | Partial exon 12 at 5′ end | 44 |
| LY2874 | Exon skipping | Partial exon 10 at 5′ end | 92 |
| | Exon skipping | Partial exon 12 at 5′ end | 44 |
| LY2881 | Intron retention | Complete Intron 14 | 437 |
| | | Complete exon 16 | 74 |
| LY3195 | Exon skipping | Partial exon 12 at 5′ end | 44 |
| LY3343 | Exon skipping | Partial exon 12 at 5′ end | 44 |
| LY3347 | Exon skipping | Partial exon 10 at 5′ end | 92 |
| | Exon skipping | Partial exon 12 at 5′ end | 44 |
| LY3353 | Exon skipping | Partial exon 12 at 5′ end | 44 |
| LY3912 | Exon skipping | Partial exon 10 at 5′ end | 92 |
| LY4228 | Exon skipping | Complete exon 12 | 161 |
| LY4235 | Intron retention | Partial intron 6 at 3′ end | 17 |
| LY4289 | Intron retention | Partial intron 6 at 3′ end | 12 |
| LY4817 | Exon skipping | Partial exon 10 at 5′ end | 11 |
| YL105 | Intron retention | Partial intron 6 at 3′ end | 12 |

The 15 mis-spliced cDNAs including 11 cDNAs that had a single event of exon skipping or intron retention and 4 cDNAs that had combination of different mis-splicing events (Table 1). The most frequent mis-splicing events were a skipping of 92 by at the 5′ end of exon 10 and 44 by at the 5′ end of exon 12, which respectively accounted for 27% and 20% of the total mis-splicing events observed among the sequenced cDNA clones.

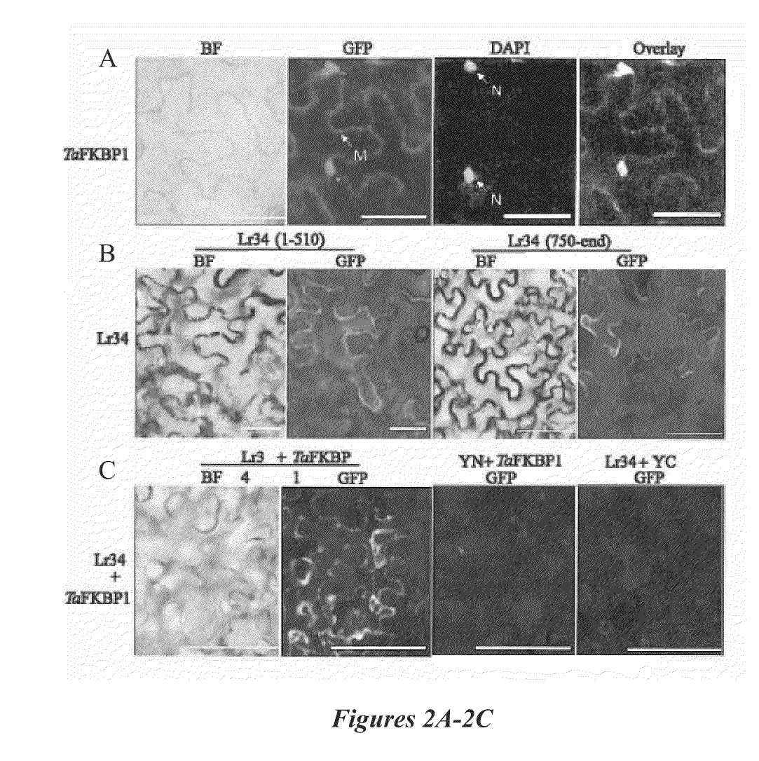

A complete Lr34 cDNA encodes a full-length functional Lr34 protein consisting of two units, each containing one cytosolic nucleotide binding domain (NBD) and one hydrophobic transmembrane domain (TD) (FIG. 1J) [2,26]. All of the mis-spliced cDNAs transcripts observed in this study resulted in a shift of the Lr34 open reading frame thus a premature stop codon, and were deduced to encode incomplete length proteins (FIG. 1C to I-1), but with one exception that was deduced to have an insertion of three amino acids in the full length protein due to retention of 12 by at the 3′ end of intron 6 (FIG. 1I-2).

A natural mutation occurs in exon 22, resulting in non-functional protein to leaf rust, due to a premature stop codon and the lack of 185 amino acids at the C terminal in the Lr34 protein of Jagger [22]. The deduced proteins from the mis-spliced cDNAs (FIG. 1C to I-1) were even shorter in length than the Jagger Lr34 protein (FIG. 1J) and did not include the 2^{nd} NBD-TD unit present in the functional protein (FIG. 1K); hence, they are unlikely to be functional in resistance to leaf rust.

To validate the hypothesis that the presence of the 2^{nd} NBD-TD unit is essential to Lr34 protein function, we first used Lr34 as bait to screen a yeast-two hybrid (Y2H) library from 2174, and identified several positive clones (not shown), including TaFKBP1 that is a peptidyl-prolyl cis-trans isomerase belonging to the immunophilin protein family [27]. Using the tobacco BiFC assays [28], we confirmed that there is a direct binding of Lr34 with TaFKBP1. As shown in FIG. 2A, when the TaFKBP1 alone was expressed, enriched yellow fluorescent signals were also localized on cytoplasma membranes and the nucleus. When an Lr34 fragment alone was expressed, enriched yellow fluorescent signals representing the Lr34 protein were localized mainly on cytoplasma membranes (FIG. 2B). Yellow fluorescence resulting from a direct interaction between Lr34 and TaFKBP1 was observed in the cytoplasmic membranes with a fluorescent microscope, only when Lr34 and TaFKBP1 were simultaneously expressed in the same cell (FIG. 2C).

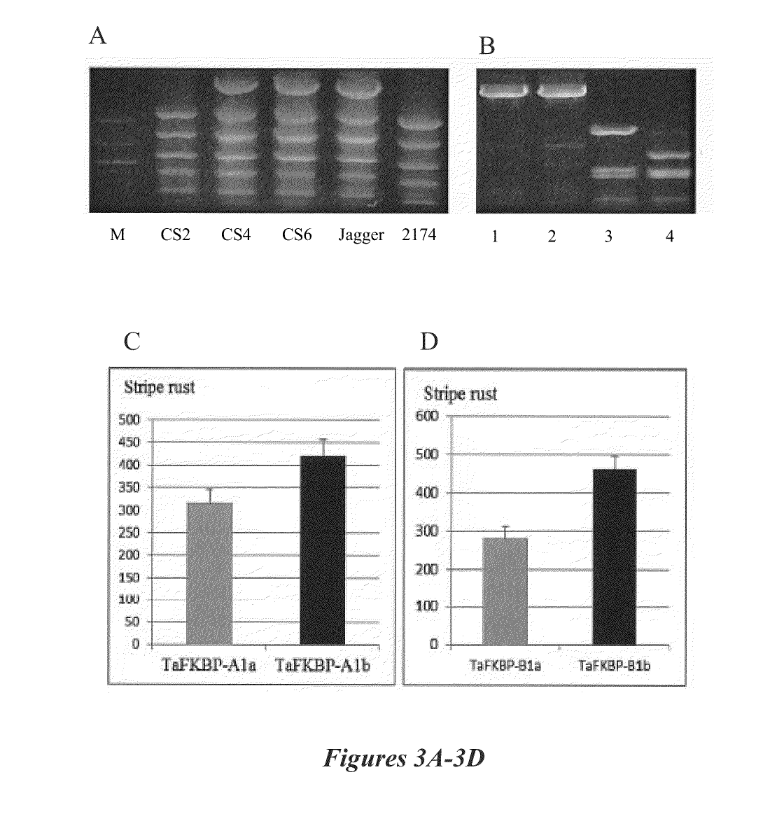

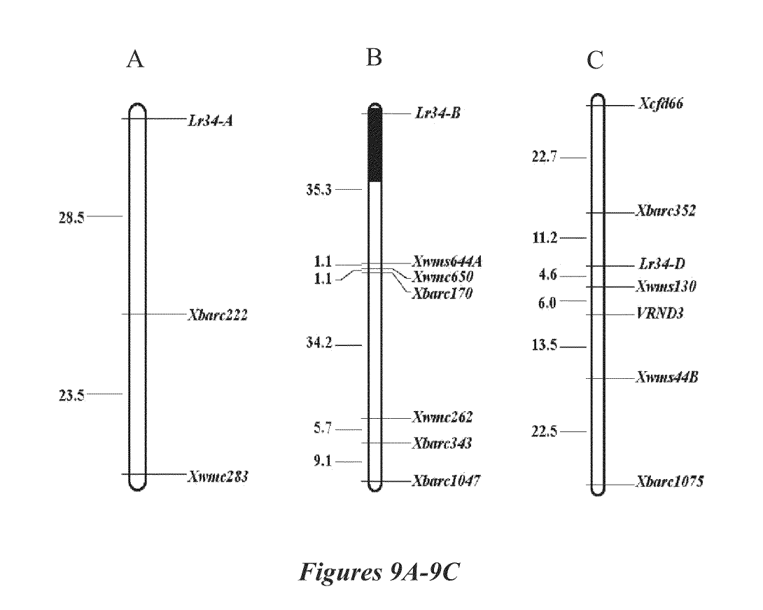

PCR markers were developed to map two of the three homoeologous genes encoding TaFKBP-A1 (FIG. 3A) and TaFKBP-B1 (FIG. 3B). No difference was found between the Jagger and 2174 alleles in the complete gene and 1,150 by upstream region from the start codon for translation of TaFKBP-D1 (KC928249). Allelic variation in TaFKBP-A1 between the Jagger allele (KC928246) and the 2174 null allele identified by the same primer was mapped to chromosome 1A (FIG. 8A) and showed genetic effects on reactions to stripe rust (p<0.01) (FIG. 3C). Allelic variation in TaFKBP-B1 between the Jagger allele (KC928247) and the 2174 allele (KC928248) was mapped to chromosome 1B (FIG. 8B) in a region including Xwmc419 that was reportedly linked with the stripe resistance gene YrC191 [29], and showed genetic effects on reactions to stripe rust (p<0.001) (FIG. 3D). The genetic effects of these TaFKBP1 genes on stripe rust were calculated using phenotypic data from in the same Jagger×2174 RIL population that was inoculated with PST-114 and PST-116 in Washington State for two years/locations. For both TaFKBP-A1 and TaFKBP-B1, Jagger had a resistant allele whereas 2174 had a susceptible allele. The allelic variation in Lr34 between the Jagger and 2174 alleles was also observed to have association with adult-plant resistance against the stripe rust race CYR32 when the same Jagger×2174 RIL population was tested in China [30]. These results indicated that TaFKBP1 and Lr34, through their physical interaction, function in the same pathway against stripe rust pathogens in wheat.

Next we attempted to demonstrate that plant resistance can be increased by increasing Lr34 transcript levels in transgenic wheat. Winter wheat ‘Jagger’ and spring wheat ‘Bobwhite’ are two cultivars that are typically used as host plants to generate transgenic wheat, but Jagger has been identified to have a susceptible allele caused by a point mutation [22] and Bobwhite was reported to have a susceptible allele [23].

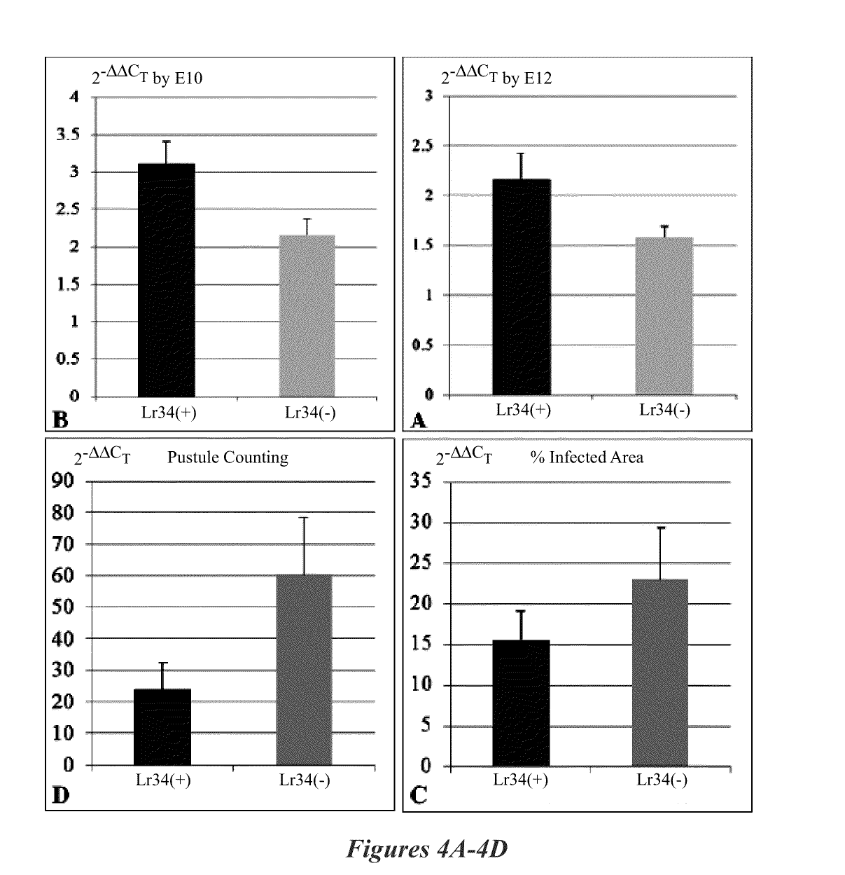

We tested wheat cultivar 2174, which contains a native, wild-type lr34 resistance gene, for its transformability and found that it possessed enhanced regenerative ability from embryo tissue. A functional Lr34 cDNA from 2174 was cloned into an over-expression vector pHAC20 and transformed back into this cultivar, and four transgenic plants were obtained. When primers were used to amplify cDNA including exon 10 and exon 12, the levels of correctly spliced transcripts increased by 44.4% (FIG. 4A) and 37.3% (FIG. 4B) respectively, in the T₂ population of the 13-5 line in transgenic wheat compared to non-transformed 2174 plants. The T₂ transgenic plants produced significantly lower pustule numbers (FIG. 4C) and a lower percentage of infected area on flag leaves (FIG. 4D) compared with the non-transformed plants. The T₂ transgenic plants were inoculated using the same leaf rust bulk isolate collection that was virulent to the RILs tested in the field [22].

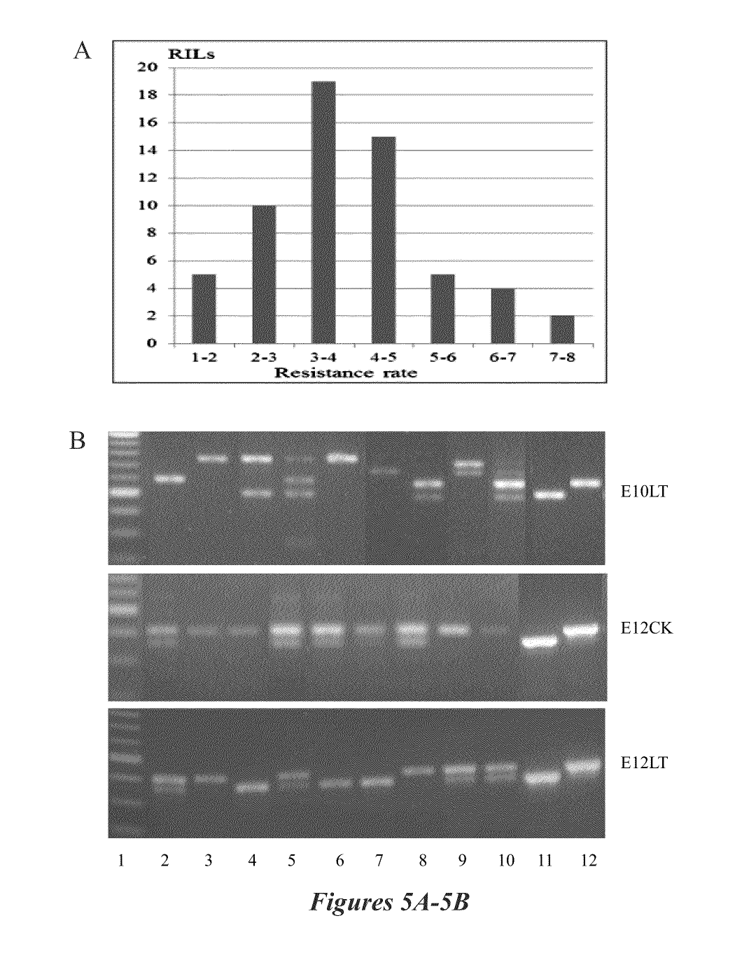

We further examined how mis-spliced Lr34 transcripts were influenced by environment and genotype by observing PCR products in an agarose gel. There are sixty lines in the mapping population of 96 Jagger×2174 RILs that were identified to have the 2174 resistant allele of Lr34, and we selected 9 representative of the variability in leaf rust reaction to determine the occurrence of mis-spliced Lr34 transcripts (FIG. 5A).

When tested with primers Lr34-DMS-F2 and Lr34-DMS-R2 for detecting partial exon 10 skipping, the proportion of mis-spliced transcripts was similar among RILs (FIG. 5B E10CK). When the plants were treated with low temperature for vernalization, however, the proportion of the mis-spliced transcripts varied from undetectable mis-splicing (lane 2, FIG. 5B E10LT) to completely mis-spliced transcripts (lane 3, FIG. 5B E10LT). Furthermore, new patterns of mis-spliced transcripts were observed in the agarose gel. Sequencing results of PCR products from two bands which migrated higher than expected (lane 9, FIG. 5B E10LT) indicated that one had retained intron 9 and the other had retained both introns 9 and 10.

When tested with primers Lr34-DMS-F3 and Lr34-DMS-R3 for detecting partial exon 12 skipping, the proportion of mis-spliced transcripts was similar among RILs at the seedling stage (FIG. 5B E12CK). When the plants were treated with low temperature for vernalization, the proportion of the mis-spliced transcripts varied widely from undetectable mis-splicing (lanes 2 and 7, FIG. 5B E12LT) to completely mis-spliced transcripts (lanes 3, 5 and 6, FIG. 5B E12LT). No new pattern of mis-spliced transcripts was visible in the agarose gel.

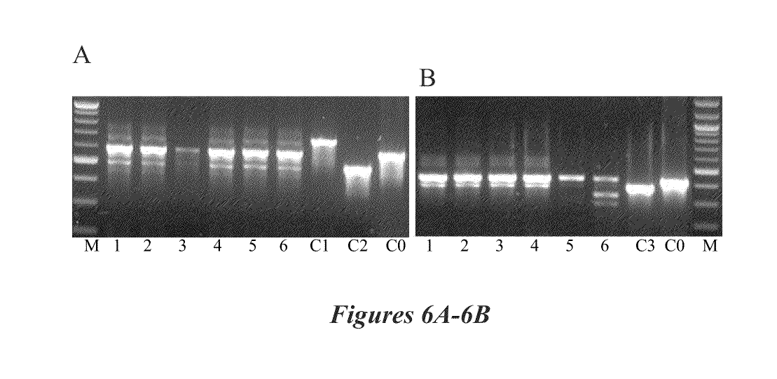

The mis-splicing of Lr34 transcripts existed not only in RILs carrying the 2174 allele but also in all of six wheat cultivars tested (FIG. 6), indicating that the biological phenomenon was not unique in 2174. The proportion of mis-spliced Lr34 transcripts was relatively stable in adult plants (FIG. 6) in these 6 cultivars compared with seedlings in the same cultivar 2174 (FIGS. 5A and 5B).

The alternative splicing events observed in the same Lr34 gene from the wheat cultivar 2174 could be interpreted using common patterns described previously, including skipped or cassette exon (CE), intron retention (IR), alternative 3′ splicing sites (A3SS), as well as alternative 5′ splicing sites (A5SS) [31]. Since a mis-spliced transcript with low expression level could not be detected in cDNA clones sequenced, it is anticipated that many more splicing events exist in Lr34 transcripts. The mis-splicing events occurring in the region from exon 1 to exon 3 were also observed in Thatcher isogenic lines in Thatcher isogenic lines [2], though it was not known how the transcripts were spliced out. In an analysis of ESTs deposited in GenBank, we found only two Lr34 ESTs in the databases, both of which are from the Chinese wheat cultivar Mingxian169. One was mis-spliced due to the presence of the complete intron 6 as well as the absence of 92-bp at the 5′ end of exon 10 and 44-bp at the 5′ end of exon 12 (GenBank accession number, GU929206), and the other was mis-spliced due to the presence of the complete intron 7 as well as the absence of 44-bp at the 5′ end of exon 12 (GU929207). Obviously, the phenomenon of mis-spliced transcripts in Lr34 in hexaploid wheat is common, but it has gone undetected in previous studies.

When a resistant Lr34 gene was artificially mutated by EMS and the mutants that lead to either alternative splice sites or mis-spliced cDNAs resulted in frame shifts, or premature stop codons, the mutant plants showed reduced resistance to leaf rust [2]. This previous study on the artificial mutant demonstrated the mis-splicing phenomenon at the DNA level. The present study demonstrated that even if a cultivar has a functional Lr34 gene, the majority of its transcripts are mis-spliced, providing a molecular understanding of mechanisms underlying the partial resistance conferred by Lr34 at the transcriptional level.

Almost all of the deduced proteins from the mis-spliced Lr34 transcripts are shorter than the truncated protein encoded by the Jagger Lr34 allele, and they thus are unlikely to have function for resistance to leaf rust or stripe rust races previously tested. However, the possibility cannot be excluded that the proteins of various lengths deduced from the mis-spliced Lr34 transcripts may contribute to resistance against powdery mildew or barley yellow dwarf virus. Protein diversity associated with different lengths generated from a single gene may relate functionally to the precise connections made by many different types of pathogens [32,33]. Functional identification of a specific Lr34 isoform by using transgenic wheat may provide clues as to whether the mis-spliced Lr34 transcripts are involved in resistance to multiple pathogens.

In this study, Lr34 was discovered to bind directly with TaFKBP1 in wheat. In Arabidopsis, FKBP42, which is a 42 kDa FK506-binding protein (FKBP42) or immunophilin protein and may be involved in protein folding and trafficking, was reported to have direct interaction with AtABCB1 that is an ABC transporter [27] in a yeast two-hybrid screen, and the interaction was confirmed using the BRET (Bioluminescence Resonance Energy Transfer) technique [34.35]. These studies support that the FKBP proteins and ABC transporters have a direct binding relationship and thus function in the same resistance pathway at the protein level in plant species. The genetic effect of TaFKBP1 on stripe rust reactions also support that TaFKBP1 functions in the resistance pathway in wheat.

The occurrence of multiple mis-spliced Lr34 transcripts in a cultivar carrying the resistant allele suggests that Lr34 did not provide full functional potential in this cultivar, and thus the resistance of this cultivar can be increased by expressing Lr34 transcripts in transgenic wheat. Winter wheat ‘Jagger’ and spring wheat ‘Bobwhite’ are two cultivars that are typically used as host plants to generate transgenic wheat, but Jagger has been identified to have a susceptible allele caused by a point mutation [30], and Bobwhite was reported to have a susceptible allele [23]. When genomic DNA of Lr34 with its own promoter was transformed to Bobwhite, the transgenic wheat showed similar resistance to leaf rust as natural wheat that carries an endogenous Lr34 gene [23]. This recent study validated Lr34 function but cannot be used to interpret the mechanism underlying the partial resistance conferred by Lr34.

The occurrence of multiple Lr34 mis-splicing events in a single RIL made it impossible to make any association between the phenotype and a specific mis-splicing event among the RILs, because a highly susceptible reaction to leaf rust could be a consequence of different mis-splicing events. The sensitivity of Lr34 splicing to low temperature made higher proportion and more forms of mis-spliced transcripts in seedlings, compared with adult plants. Resistance of wheat to rust diseases is sensitive to temperature [36-38]. Lr34 is a gene that provides durable and non-specific adult plant resistance, and its resistance phenotype at seedling stage is influenced by temperature [39]. The variable effects of Lr34 were most likely due to the temperature sensitivity of this gene. This study provided the first molecular evidence for the differential resistance conferred by Lr34 between seedling and adult plants. The findings in this study lead to a novel strategy of breeding cultivars resistant to rust pathogens. The mis-splicing events resulted in non-functional forms of the Lr34 protein for resistance to the rust diseases in wheat, which is a natural biological event that might be ameliorated by an understanding of mechanisms underlying mis-splicing events. Splicing of a gene is performed by an RNA and protein complex known as the spliceosome, a process regulated by a group of proteins or auxiliary elements known as exon splicing enhancers and silencers (ESEs and ESSs) and intron splicing enhancers and silencers (ISEs and ISSs) [40]. Enhancers can activate adjacent splice sites or antagonize silencers, whereas silencers can repress splice sites or enhancers. The level of resistance conferred by a quantitative gene can be increased to the level of resistance conferred by a qualitative gene by eliminating or mutating regulators in the spliceosome that cause mis-splicing events in wheat.

Materials and Methods

Sequences of mis-spliced Lr34 transcripts from 2174 carrying a resistance allele.

Winter wheat cultivar 2174 that has been experimentally confirmed to have a resistant allele at Lr34 on homoeologous chromosome was used to investigate its expression in leaves collected from seedlings at 3^{rd} leaf stage and plants that were treated with low temperature for 3 or 6 weeks. The total RNA was isolated using TRIZOL and RNAs were reverse transcribed by SuperScript II reverse Transcriptase (Invitrogen, Grand Island, N.Y., USA). The paired primers Lr34-Exp-F1 and Lr34-Exp-R1 (Table 2) were designed to amplify complete cDNA of Lr34 including 36 by at the 5′ UTR and 22 by at the 3′ UTR. PCR was performed using LongAmp Taq DNA polymerase (New England Biolabs, Ipswich, Mass., USA) The PCR products of ˜4.2 kb cDNAs were then run at a 1% low melting gel, purified by Wizard SV Gel and PCR Clean-Up system (Promega, Madison, Wis., USA), and cloned into pGEM-T vector (Promega, Madison, Wis., USA). Plasmid DNAs from single clones were completely sequenced. Since the Lr34-Exp-F1 and Lr34-Exp-R1 primers also amplified cDNA of Lr34-B (on homoeologous genome B) in hexaploid wheat, only the sequences from Lr34 were analyzed in this study as outlined below.

Identification of Proteins Interacting with Lr34

The ‘Matchmaker™’ Library Construction & Screening System (Clontech, USA) was used to construct a Y2H ‘prey’ library for 2174 RNA that was extracted from pooled samples of young leaves and apices from vernalized plants for 1, 2, and 3 weeks [41]. Lr34 was used as ‘bait’ to screen the ‘prey’ library. The full length Lr34 was not expressed by the pGBKT7 BD vector; therefore, we expressed a protein fragment encoding the second NBD-TD unit of Lr34. The resulting prey constructer was used to search the Y2H library constructed in GAL4 AD vectors. The mating-resultant zygotes cells were spread on the medium containing SD/-Ade/-His/-Leu/-Trp to select for putative positive two-hybrid interactions. Approximately 50 million transformats were screened, and a total of 21 positive clones were obtained from the 2174 library. These 21 clones were sequenced and annotated, and the resulting 13 genes including TaFKBP1 (Supplementary Table 2). The protein interactions between Lr34 and TaFKBP1 protein observed from the putative positive colonies in the Y2H assays were validated in bimolecular fluorescence complementation (BiFC) analyses.

In Vivo BiFC Interaction between Lr34 and TaFKBP1

The full length Lr34 was not expressed by the pEG101-YFP vector (258) or a series of BiFC vectors [42]; therefore, two Lr34 fragments encoding amino acids from positions 1 to 510 included in the first NBD-TD unit of Lr34 and from positions 750 to 1401 included in the second NBD-TD unit of Lr34 were tested. For TaFKBP1, its complete cDNA was tested by using primers FKBP1-BiFC1-F1 and FKBP1-BiFC1-R1. For Lr34, two cDNA fragments were tested using primers Lr34-BiFCE10-F1 and Lr34-BiFCE10-R1 for the first NBD-TD unit (1-510) and primers Lr34-BiFCE12-F1 and Lr34-BiFCE12-R1 for the second NBD-TD unit (750-end). Both Lr34 and TaFKBP1 from 2174 were firstly cloned into pDONR207 with the BP cloning kit (Invitrogen), and then transferred to pEarleygate 101 (pEG101, Invitrogen) to express their proteins and to determine the physical location of a single protein in living cells of leaves of tobacco (N. benthamiana) for subcellular localization used protocol previously described [28]. The primer sequences are provided in Supplementary Table 3.

Lr34 encoding amino acids from positions 750 to 1401, as used in Y2H assays, was cloned into pEG201-YN, and TaFKBP1 was cloned into pEG202-YC. Using LR cloning (Invitrogen), Lr34(750-end) in pDONR207 was fused to the N-terminal 174 amino acid portion of YFP in the pEarleyGate201 vector (pEG201-YN) to test for in vivo interaction with TaFKBP1 fused to the C-terminal 66 amino acid portion of YFP in the pEarleyGate202 vector (pEG202-YC) [28]. The two constructs were co-transformed into plants.

A. tumefaciens strains (GV3101) carrying the BiFC constructs were used together with the p19 strain (for suppression of gene silencing of constructs) for infiltration of N. benthamiana leaves (5 weeks old). Leaf discs were cut for BiFC for imaging three days after infiltration. Images were collected on a fluorescent microscope (Olympus BX51): 460-490 nm for excitation and 510-550 nm for emission were used in the case of GFP (also called GFPA), and 320-390 nm for excitation and 430-490 nm for emission were used in the case of DAPI.

Transgenic Wheat Overexpressing Lr34

Primers Lr34-OE-F1 and Lr34-OE-R1 (Supplementary Table 3) were used to clone a complete Lr34 cDNA from 2174 into a pGEM-T vector (Promega) and then the cDNA was transferred to pAHC20 vector [43] for expression in 2174. The engineered plasmid was co-transformed in a 2:1 molar ratio with UBI:BAR into immature embryos of variety 2174 by microprojectile bombardment as described before [45] except that both shoot regeneration media and rooting media contained 1.5 mg/L bialaphos to select the transformants.

After the flag leaves emerged, transgenic plants and wild type 2174 were inoculated with leaf rust spores collected from leaves of the naturally infected cultivar Jagger [22]. Inoculated plants were kept in a dew chamber at 20-24° C. then moved to a growth chamber operating at 16 h light and 8 h dark with diurnal temperatures gradually changing from 10° C. to 15° C. Pustules numbers were counted and the percentage of leaf area producing lesions was determined by the software APS Access 2.0 on flag leaves 23 to 25 days after inoculation. The mean of pustule number and the percentage lesion area from 3 flag leaves per plant was used for comparison.

Primer Lr34-SYBR-E10F1 spanning exon 9 and exon 10 was used to pair with Lr34-SYBR-E10R2 specific to exon 10 of Lr34 to amplify a 211 by cDNA fragment. Primer Lr34E12 Lr34-SYBR-E12F2 that was specific to exon 11 of Lr34 was paired with primer Lr34-SYBR-E12R2 that covered exon 11 and exon 12 to amplify a 111 by cDNA fragment. Primers actin-F1 and actin-R1 and were used to amplify transcripts of actin as endogenous control. The Lr34 transcripts were assessed using the SYBR Green PCR Master Mix (Applied Biosystems, Life Technologies) and the Applied Biosystems 7500 Real-Time PCR Systems. All of the primer sequences are provided in Table 2.

TABLE 2
List of primers used for Lr34 mis-splicing.

| Primer name | Primer sequence (5′-3′) |
|---|---|
| Lr34-Exp-F1 | TAGCAAAGGGCGTCGATTTA (SEQ ID NO: 3) |
| Lr34-Exp-R1 | CTGTTGGAATGTCAGAACTTGC (SEQ ID NO: 4) |
| Lr34-DMS-F2^{A} | AGATGATTGTGGGCCCCGCAAGT (SEQ ID NO: 5) |
| Lr34-DMS-R2^{B} | TTACGAGTGCAATAATGGCAAGCTG (SEQ ID NO: 6) |
| Lr34-DMS-F3^{A} | CTATATGGGAGCATTATTTTTTTCCATCATG (SEQ ID NO: 7) |
| Lr34-DMS-R3^{C} | AACCATCCTGGCATGGAGGTCT (SEQ ID NO: 8) |
| Lr34-EcoRI-F1^{D} | CGGAATTCGATTACAGAACACCTACAGAAGAATATC (SEQ ID NO: 9) |
| Lr34-BamHI-R1^{D} | CGGGATCCTTACCTCTTCTGGAAATTAAGTTTCTC (SEQ ID NO: 10) |
| Lr34-BiFCE10-F1^{E} | GGGGACAAGTTTGTACAAAAAAGCAGGCTTCATGGAGGGCCTCGCAAGA |
| | GAGA (SEQ ID NO: 11) |
| Lr34-BiFCE10-R1^{F} | GGGGACCACTTTGTACAAGAAAGCTGGGTCCTGGCCTGTTTTGAAGACA |
| | TAAAC (SEQ ID NO: 12) |
| Lr34-BiFCE12-F1^{E} | GGGGACAAGTTTGTACAAAAAAGCAGGCTTCATGGATTACAGAACACCT |
| | ACAGAAGAA (SEQ ID NO: 13) |
| Lr34-BiFCE12-R1^{F} | GGGGACCACTTTGTACAAGAAAGCTGGGTCCCTCTTCTGGAAATTAAGT |
| | TTCTC (SEQ ID NO: 14) |
| FKBP1-BiFC1-F1^{D} | GGGGACAAGTTTGTACAAAAAAGCAGGCTTCATGGCAGAAACCATTGAT |
| | CTAAC (SEQ ID NO: 15) |
| FKBP1-BiFC1-R1^{F} | GGGGACCACTTTGTACAAGAAAGCTGGGTCTTTGCCCTTTCCTTTTCCC |
| | TTGC (SEQ ID NO: 16) |
| Lr34-OE-F1 | CGGGATCCATGGAGGGCCTCGCAAGAGAG (SEQ ID NO: 17) |
| Lr34-OE-R1 | CGGGATCCTTACCTCTTCTGGAAATTAAG (SEQ ID NO: 18) |
| Lr34-SYBR-E10F1^{G} | AGCTGACTTTCTTCAAGAGATC (SEQ ID NO: 19) |
| Lr34-SYBR-E10R2^{H} | TTTTTGTAGCGAATACTTATTGAATG (SEQ ID NO: 20) |
| Lr34-SYBR-E12F2^{I} | CTTCAGTCCTAAAGGTCCCTA (SEQ ID NO: 21) |
| Lr34-SYBR-E12R2^{J} | GAAACTGGCAGAAGAACCTTG (SEQ ID NO: 22) |
| actin-F1 | ATGGAAGCTGCTGGAATCCAT (SEQ ID NO: 23) |
| actin-R1 | CCTTGCTCATACGGTCAGCAATAC (SEQ ID NO: 24) |
| Lr34-X-F2 | AGAATGGTGCTGCAAACACCGG (SEQ ID NO: 25) |
| Lr34-In2-R1 | GTGTCTTATCAGTTCCATTCTCATTGATTC (SEQ ID NO: 26) |
| Lr34B-5-F1 | GCAAAGGGCGTCGATTTAAG (SEQ ID NO: 27) |
| Lr34-In2R1 | ATCAGTTCCATTTTCATTGAGTTGGT (SEQ ID NO: 28) |
| FKBP1-F2 | CGCCGCCACGGCCCTCC (SEQ ID NO: 29) |
| FKBP1-R2 | CTACATCCCATAATGTAAAATGTTTTTTGATGAG (SEQ ID NO: 30) |
| FKBP1-C1-F2 | ATGTAGGTCTGCCTAGTTGGGCG (SEQ ID NO: 31) |
| FKBP1-ABD-R1 | GCTTTAATGACAGCTCCCTGGCCAACTTC (SEQ ID NO: 32) |

Notes:
A, Specific to Lr34.
B, Covered both exon 10 and exon 11.
C, Covered both exon 12 and exon 13.
D, Italic sequences from Lr34.
E, ATG was added to translate the gene
F, The native stop codon TAA was removed to enable the YFP gene to express.
G, Covered both exon 9 and exon 10
H, Specific to exon 10 of Lr34.
I, Specific to exon 11 of Lr34.
J, Covered both exon 11 and exon 12

### Allelic Variation and Mapping of TaFKBP1

Three homoeologous TaFKBP1 genes were isolated from each of Jagger and 2174. Primers FKBP1-F2 (5′-CGCCGCCACGGCCCTCC-3′, SEQ ID NO: 33) and FKBP1-R2 (5′-CTACATCCCATAATGTAAAATGTTTTTTGATGAG-3′, SEQ ID NO: 34) were used to map FKBP-A1. The two primers amplified ˜2 kb fragment from Jagger. The PCR products were directly run in a 1% agarose gel, showing polymorphic bands from the Jagger allele, but not from 2174 allele.

Primers FKBP1-C1-F2 (5′-ATGTAGGTCTGCCTAGTTGGGCG-3′, SEQ ID NO: 35) and FKBP1-ABD-R1 (5′-GCTTTAATGACAGCTCCCTGGCCAACTTC-3′, SEQ ID NO: 36) were used to map FKBP-B1. The two primers amplified ˜1.5 kb fragment. The PCR products digested with restriction enzyme DdeI were run in a 1% agarose gel, showing polymorphic bands between the Jagger allele (750 bp) and 2174 allele (448 bp).

Accession Numbers

Sequence data from this article can be found in GenBank!EMBL databases under the following accession numbers: Jagger TaFKBP-A1 (KC928246), Jagger TaFKBP-B1 (KC928247), 2174 TaFKBP-B1 (KC928248), and Jagger and 2174 TaFKBP-A1 (KC928249).

### REFERENCES

1. Fu D, Uauy C, Distelfeld A, Blechl A, Epstein L, et al. (2009) A kinase-START gene confers temperature-dependent resistance to wheat stripe rust. Science 323: 1357-1360.
2. Krattinger S G, Lagudah E S, Spielmeyer W, Singh R P, Huerta-Espino J, et al. (2009) A putative ABC transporter confers durable resistance to multiple fungal pathogens in wheat. Science 323: 1360-1363.
3. McIntosh R A, Yamazaki Y, Devos K M, Dubcovsky J, Rogers J, et al. (2008) Catalogue of gene symbols. Mac-Gene 2008.
4. Bjarko M E, Line R F (1988) Quantitative determination of the gene action of leaf rust resistance in four cultivars of wheat, Triticum aestivum. Phytopathology 78: 457-461.
5. Caldwell R M, Roberts J J, Eyal Z (1970) General resistance (“slow-rusting”) to Puccinia recondita f. sp. tritici in winter and spring wheat. Phytopathology 60: 1287.
6. Das M K, Rajaram S, Kronstad W E, Mundt C C, Singh R P (1993) Associations and genetics of three components of slow rusting in leaf rust of wheat. Euphytica 68: 99-109.
7. Dyck. P L, Samborski D L, Anderson R G (1966) Inheritance of adult-plant leaf rust resistance derived from the common wheat varieties Exchange and Frontana. Can J Genet Cytol 8: 665-671.
8. Kolmer J, Chen X, Jin Y (2009) Diseases which challenge global wheat production—the wheat rusts. In: Carver B F (ed.) Wheat: Science and Trade. Wiley-Blackwell, Iowa. pp. 89-124.
9. Parlevliet J E, Zadoks J C (1977) The integrated concept of disease resistance; a new view regarding horizontal and vertical resistance in plants. Euphytica 26: 5-21.
10. Rubiales D, Niks R E (1995) Characterization of Lr34, a major gene conferring nonhypersensitive resistance to wheat leaf rust. Plant Dis 79: 1208-1212.
11. Singh R P, Huerta-Espino J, Rajaram S (2000) Achieving near-immunity to leaf and stripe rusts in wheat by combining slow rusting resistance genes. Acta Phytopathol. Entomol. Hung. 35: 133-139.
12. Flor H H (1971) Current status of the gene-for-gene concept. Annu Rev Phytopathol 9: 275-296.
13. Lillemo M, Asalf B, Singh R P, Huerta-Espino J, Chen X M, et al. (2008) The adult plant rust resistance loci Lr34/Yr18 and Lr46/Yr29 are important determinants of partial resistance to powdery mildew in bread wheat line Saar. Theor Appl Genet 116: 1155-1166.
14. Singh R P, Trethowan R (2007) in Breeding Major Food Staples, M. Kang, P. M. Priyadarshan, Eds. (Blackwell, Ames, Iowa, 2007), pp. 109-140.
15. Kröner A, Hamelin G, Andrivon D, Val F (2011) Quantitative resistance of potato to Pectobacterium atrosepticum and Phytophthora infestans: integrating PAMP-triggered response and pathogen growth. PLoS One 6: e23331.
16. Vergne E, Grand X, Ballini E, Chalvon V, Saindrenan P, et al. (2010) Preformed expression of defense is a hallmark of partial resistance to rice blast fungal pathogen Magnaporthe oryzae. BMC Plant Biol 10: 206-223.
17. Kolmer J A, Garvin D F, Jin Y (2011) Expression of a Thatcher wheat adult plant stem rust resistance QTL on chromosome arm 2BL is enhanced by Lr34 Crop Sci. 51: 526-533.
18. Lagudah E S, Krattinger S G, Herrera-Foessel S, Singh R., Huerta-Espino J, et al. (2009) Gene-specific markers for the wheat gene Lr34/Yr18/Pm38 which confers resistance to multiple fungal pathogens. Theor Appl Genet 118: 889-898.
19. Singh R P (1993) Genetic association of gene Bdv1 for tolerance to barley yellow dwarf virus with genes Lr34 and Yr18 for adult plant resistance to rusts in bread wheat. Plant Dis 77: 1103-1106.
20. Suenaga, K., Singh, R. P., Huerta-Espino, J., and William, H. M. (2003). Microsatellite markers for genes Lr34/Yr18 and other quantitative trait loci for leaf rust and stripe rust resistance in bread wheat. Phytopathology 93: 881-890.
21. Bossolini E, Krattinger S G, Keller B (2006) Development of simple sequence repeat markers specific for the Lr34 resistance region of wheat using sequence information from rice and Aegilops tauschii. Theor Appl Genet 113: 1049-1062.
22. Cao S, Carver B F, Zhu X, Fang T, Chen Y, et al. (2010) A single-nucleotide polymorphism that accounts for allelic variation in the Lr34 gene and leaf rust reaction in hard winter wheat. Theor Appl Genet 121: 385-392.
23. Risk J M, Selter L L, Krattinger S G, Viccars L A, Richardson T M, et al. (2012) Functional variability of the Lr34 durable resistance gene in transgenic wheat. Plant Biotech J 10: 477-87.
24. Dakouri A, McCallum B D, Walichnowski A Z, Cloutier S (2010) Fine-mapping of the leaf rust Lr34 locus in Triticum aestivum (L.) and characterization of large germplasm collections support the ABC transporter as essential for gene function. Theor Appl Genet 121: 373-84.
25. Schnurbusch T, Paillard S, Schori A, Messmer M, Schachermayr G, et al. (2004) Dissection of quantitative and durable leaf rust resistance in Swiss winter wheat reveals a major resistance QTL in the Lr34 chromosomal region. Theor Appl Genet 108: 477-484.
26. Krattinger, S. G., Lagudah, E. S., Wicker, T., Risk, J. M., Ashton, A. R., Selter, L. L., Matsumoto, T., and Keller, B. (2011). Lr34 multi-pathogen resistance ABC transporter: molecular analysis of homoeologous and orthologous genes in hexaploid wheat and other grass species. Plant J 65: 392-403.
27. Geisler M, Girin M, Brandt S, Vincenzetti V, Plaza S, et al, (2004) Arabidopsis immunophilin-like TWD1 functionally interacts with vacuolar ABC transporters. Mol Biol Cell 15: 3393-3405.
28. Lu Q, Tang X, Tian G, Wang F, Liu K, et al. (2010) Arabidopsis homolog of the yeast TREX-2 mRNA export complex: components and anchoring nucleoporin. Plant J 60: 259-270.
29. Ren Q, Liu H J, Cen Y, Xu S C, He M Z, et al. (2010). Molecular Tagging of a Stripe Rust Resistance Gene in a Triticum durum-Aegilops squarrosa Synthetic Wheat CI191. ACTA AGRONOMICA SINICA. 36: 721-727
30. Fang T, Campbell K G, Liu Z, Chen X, Wan A, et al. (2011). Stripe rust resistance in the wheat cultivar Jagger is due to Yr17 and a novel resistance gene. Crop Sci. 51: 2455-2465.
31. Koscielny G, Le Texier V, Gopalakrishnan C, Kumanduri V, Riethoven J J, et al. (2009) ASTD: The alternative splicing and transcript diversity database. Genomics 93: 213-220.
32. Kriventseva E V, Koch I, Apweiler R, Vingron M, Bork P, et al. (2003). Increase of functional diversity by alternative splicing. Trends Genet 19: 124-128.
33. Matlin A J, Clark F, Smith C W J (2005) Understanding alternative splicing: towards a cellular code. Nature Rev Mol Cell Biol 6: 386-398.
34. Bailly A, Sovero V, Geisler M (2006). The TWISTED DWARF's ABC: how immunophilins regulate auxin transport. Plant Signaling Behav 1: 277-280.
35. Bouchard R, Bailly A, Blakeslee J J, Oehring S C, Vincenzetti V, et al. (2006) Immunophilin-like TWISTED DWARF1 modulates auxin efflux activities of Arabidopsis P-glycoproteins. J Biol Chem 281: 30603-30612.
36. Dyck P L, Samborski D J (1982) The inheritance of resistance to Puccinia recondita in a group of common wheat cultivars. Can J Genet Cytol 24: 273-283.
37. Pretorius Z. A., F. H. J. Rijkenberg and R. D. Wilcoxon, 1988. Effects of growth stage, leaf position and temperature on adult-plant resistance of wheat infected by Puccinia recondita f. sp. tritici. Plant Pathol 37: 36-44.
38. Statler G D, Christianson T, (1993) Temperature studies with wheat leaf rust. Canadian J Plant Pathol 15: 97-101.
39. Singh R P (1992) Genetic association of leaf rust resistance gene Lr34 with adult-plant resistance to stripe rust in bread wheat. Phytopathology 82: 835-838.
40. Black D L (2003) Mechanisms of alternative pre-messenger RNA splicing. Annual Rev of Biochem 72: 291-336.
41. Cao S, Yan L (2013) Construction of a high-quality Yeast two-hybrid (Y2H) library and its application in identification of interacting proteins with key vernalization regulator TaVRN-A1 in wheat. BMC Res Note 6: 81-86.
42. Waadt, R., Schmidt, L. K., Lohse, M., Hashimoto, K., Bock, R., and Kudla, J. (2008). Multicolor bimolecular fluorescence complementation reveals simultaneous formation of alternative CBL/CIPK complexes in planta. Plant J. 56: 505-516.
43. Christensen A H, Quail P H (1996) Ubiquitin promoter-based vectors for high level expression of selectable and/or screenable marker genes in monocotyledonous plants. Transgenic Res. 5: 213-218.
44. Okubara P A, Blechl A E, McCormick S P, Alexander N J, Dill-Macky R, et al. (2002) Engineering deoxynivalenol metabolism in wheat through the expression of a fungal trichothecene acetyltransferase gene. Theor Appl Genet 106: 74-83.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the spirit of this invention as defined by the appended claims.

The contents of all patents, patent applications and publications cited herein are hereby incorporated by reference in entirety.

What is claimed is:

1. A transgenic 2174 wheat cultivar that contains a native Lr34 resistance gene and is genetically transformed to contain and express a cDNA sequence lacking introns and encoding an Lr34 resistance transgene.

2. The transgenic 2174 wheat cultivar of claim 1, wherein said cDNA sequence is operably linked to a heterologous promoter.

3. The transgenic2174 wheat cultivar of claim 1, wherein said cDNA sequence has a nucleotide sequence as represented in SEQ ID NO: 1.

4. The transgenic 2174 wheat cultivar of claim 1, wherein said transgenic 2174 wheat cultivar is resistant to a fungal disease selected from the group consisting of leaf rust, stripe rust, powdery mildew, and barley yellow dwarf virus.

5. A method of increasing resistance to a fungal disease in a 2174 wheat cultivar that contains a native Lr34 resistance gene, comprising
genetically transforming said 2174 wheat cultivar to contain and express a cDNA sequence lacking introns and encoding an Lr34 resistance transgene.

6. The method of claim 5, wherein said cDNA sequence is operably linked to a heterologous promoter.

7. The method of claim 5, cDNA sequence has a nucleotide sequence as represented in SEQ ID NO: 1.

8. The method of claim 5, wherein said fungal disease is selected from the group consisting of leaf rust, stripe rust, powdery mildew, and barley yellow dwarf virus.
